SEQUENCE LISTING (24 sequences)

SEQ ID NO: 1 - DNA, 26 bp, Influenza virus
ggggtagccccattgcatttgggtaa

SEQ ID NO: 2 - DNA, 26 bp, Influenza virus
aacagttgctgtaggctttgctgcgt

SEQ ID NO: 3 - DNA, 26 bp, Influenza virus
tggattctttgtctgcagcgtaccca

SEQ ID NO: 4 - DNA, 24 bp, Influenza virus
ccgctgcttagtttgactgggtca

SEQ ID NO: 5 - DNA, 22 bp, Influenza virus
atgttgaacgaaacttccgctg

SEQ ID NO: 6 - DNA, 21 bp, Influenza virus
tgtggagttgataaggggaag

SEQ ID NO: 7 - DNA, 23 bp, Influenza virus
tgaccagtcggcaatctcatagt

SEQ ID NO: 8 - DNA, 24 bp, Influenza virus
gggtcattcggtcggggattgtct

SEQ ID NO: 9 - DNA, 13 bp, Influenza virus
ttacccaaatgca

SEQ ID NO: 10 - DNA, 13 bp, Influenza virus
acgcagcaaagcc

SEQ ID NO: 11 - DNA, 13 bp, Influenza virus
tgggtacgctgca

SEQ ID NO: 12 - DNA, 12 bp, Influenza virus
tgacccagtcaa

SEQ ID NO: 13 - DNA, 11 bp, Influenza virus
cagcggaagtt

SEQ ID NO: 14 - DNA, 11 bp, Influenza virus
cttccccttat

SEQ ID NO: 15 - DNA, 12 bp, Influenza virus
actatgagattg

SEQ ID NO: 16 - DNA, 12 bp, Influenza virus
agacaatccccg

SEQ ID NO: 17 - DNA, 13 bp, Influenza virus
atggggctacccc

SEQ ID NO: 18 - DNA, 13 bp, Influenza virus
tacagcaactgtt

SEQ ID NO: 19 - DNA, 13 bp, Influenza virus
gacaaagaatcca

SEQ ID NO: 20 - DNA, 12 bp, Influenza virus
actaagcagcgg

SEQ ID NO: 21 - DNA, 11 bp, Influenza virus
tcgttcaacat

SEQ ID NO: 22 - DNA, 10 bp, Influenza virus
caactccaca

SEQ ID NO: 23 - DNA, 11 bp, Influenza virus
ccgactggtca

SEQ ID NO: 24 - DNA, 12 bp, Influenza virus
accgaatgaccc

(19) United States
(12) Patent Application Publication
(10) Pub. No.: US 2019/0119731 A1
(43) Pub. Date: Apr. 25, 2019

(54) NANOPROBE SANDWICH ASSAY FOR NUCLEOTIDE SEQUENCE DETECTION

(71) Applicant: The Hong Kong Polytechnic University, Hong Kong (CN)

(72) Inventors: Ming-Kiu TSANG, Hong Kong (CN); Yuen-Ting WONG, Hong Kong (CN); Jianhua HAO, Hong Kong (CN)

(21) Appl. No.: 15/792,156

(22) Filed: Oct. 24, 2017

(51) Int. Cl. C12Q 1/68

(52) U.S. Cl. CPC C12Q 1/6818

(57) ABSTRACT

The invention relates to nucleotide sequence detection based on upconversion nanoprobes and quenching nanoprobes in a sandwich assay.

### FIELD OF THE INVENTION

The invention relates to an assay for nucleotide sequence detection.

### BACKGROUND OF THE INVENTION

Seasonal influenza has been a threat to Hong Kong because of the rapid mutation of influenza viruses, the death toll in 2017 also broke the record in summer. The serious outbreak was due to the mutation of the H3N2 virus gene. Considering the rapid mutation and spread, fast and accurate screening holds the key for effective suppression of epidemic. The reverse transcription polymerase chain reaction (RT-PCR) and enzyme-linked immunosorbent assay (ELISA) are two gold standards in conventional clinics. In the flow of RT-PCR, the viral ribonucleic acid (RNA) is transcripted into deoxyribonucleic acid (DNA) for amplification through thermal cycling. This process required a considerable amount of time and sophisticated personnel for the DNA amplification. Therefore, the whole process might take up to one to three days for accurate results. In recent year, the real time polymerase chain reaction (qPCR) had been developed to increase the efficiency of DNA amplification and detection process. qPCR involves the use of fluorescent dyes probe to monitor the number of DNA molecules via observing the optical signal change. Although the dyes can be excited by light emitting diodes, the intrinsic broad emission peak induces cross-talking with the excitation source. As a result, this interferes the detection signal or leads to false positive results. On the other hand, ELISA relies on the antibody-antigen interaction for detection; the readout format can be absorbance or fluorescence. This technique is relatively cheaper and easier than PCR techniques, but the limit of detection is inferior to PCRs and the washing steps are laborious. Nowadays, the sandwich assay format had been widely used in ELISA assays and researches in optical sensing technologies. However, the optical probes still suffer from non-idealities, such as high energy excitation and cross-talking.

The present and well-known technology for DNA oligonucleotide (oligo) detection is reverse transcription polymerase chain reaction (RT-PCR) and enzyme-linked immunosorbent assay (ELISA). The techniques are essential for identifying virus genes in one sample. Firstly, the RT-PCR technique is a genetic diagnostic technique based on cloning expressed genes by reverse transcribing the RNA of virus into its DNA complement and amplification of the complement DNA (c-DNA) via thermo-cycling in a thermos cycler. This technology involves the sophistically-designed primers for efficient amplification via nucleic acid hybridization. The readout is done by using gel electrophoresis. The whole process may require 1-3 days for accurate results. The ELISA technique is a solid-state colorimetric immunoassay, which is based on antibody-antigen interaction via the viruses surface protein. Initially, the antigen is anchored on the substrate and a specific type of antibody linked with enzyme is added to the substrate. The interaction of the antigen and the antibody will form a complex to produce color change. As a result, the antibody expressed on the surface of virus can be identified by such technique. However, PCR requires well-trained personnel for operating the thermocycler and the amplification process is relatively time-consuming. The amplification steps are prone to contamination during successive steps. On the other hand, ELISA kits are commercialized and available from many suppliers. The kits consist of necessary chemicals and substrate for testes. However, the procedures of ELISA are laborious and the limit of detection is relatively low (nanomolar range). Owing to these shortcomings, the quest for searching sensitive and quick diagnostic assays is still on-going.

In recent years, luminescent assays are drawing attention because of their high sensitivity and the ease of making portable devices for on-site biodetections. Luminescent assays are divided into homogeneous and heterogeneous assays. Homogeneous assays are liquid phase test, and they are usually performed in micro-centrifuge tubes and simple mixing steps are required to observe the results. On the other hand, heterogeneous assays are more sensitive than homogeneous assay because of the higher binding affinity between the probe and analyte. One of the key features is the use of a solid phase substrate for detection. The results in both assays can be interpreted by using a portable light source and simple optical detectors, such as CMOS or CCDs. Therefore, they are much simpler than PCR and ELISA techniques. Nowadays, downconversion (DC) or downshifting (DS) luminescence-based assays are being reported for rapid luminescent detections. However, such luminescence mechanisms require the use of high energy light sources, such as ultraviolet (UV). It is a common knowledge that UV is harmful to DNAs and it will destroy chemical oligo chain backbones. Moreover, UV will induce autofluorescence, which will contribute to false-positive detection signals. As a result, upconversion luminescence (UCL) assays are developed to overcome the above-mentioned drawbacks. UCL is a unique luminescent phenomenon that involves sequential absorption of lower energy photons to emit a higher energy photon. In this regard, the low energy excitation can reduce the photodamage to biological samples to a minimum. Moreover, it is easier to distinguish the luminescent detection signal because of the large anti-stoke shift and the invisible near infrared (NIR) excitation. Despite UCL requires the use of lasers, the availability of cheap and portable diode lasers has overcome the issue.

The upconversion nanoparticles (UCNPs) can be obtained by hydrothermal method. The advantages are simplicity and ease of manipulation because water dispersible UCNPs with amine (NH₂) surface is readily obtained via a one-step hydrothermal method. However, it is relatively time consuming, requiring about 24 h of reaction time for completion, and the resultant NH₂-UCNPs are not regular in shape.

The UCNPs BaGdF₅:Yb/Er has been disclosed for homogeneous detection of Avian Influenza Virus H7 subtype (Small 2014, 10, 2390-2397) and heterogeneous detection of Ebola virus oligonucleotide (ACS Nano 2016, 10, 598-605). The UCNP of BaGdF₅:Yb/Er was synthesized by hydrothermal method, and the detection scheme was suitable for single target only. Since the emission intensity of BaGdF₅:Yb/Er is weak and the nanoparticle is not dispersing very well in water, it is difficult to control their position during the fabrication of the microarray for simultaneous detection of multi-targets.

In addition, structural engineering of core-shell upconversion nanoparticles (csUCNPs) has emerged as a powerful means to integrate functionalities and regulate the complex interplay of lanthanide interactions. The csUCNPs can be obtained by thermal decomposition method and co-precipitation synthesis.

The core-shell NaGdF₄:Yb/Er@NaGdF₄:Yb/Nd has been disclosed for in vitro and in vivo imaging (ACS Nano 2013, 7, 7200-7206), prepared by thermal decomposition method. The limitations of thermal decomposition method disclosed in the ACS Nano 2013 paper mainly arise from the synthetic route that involves the use of excessive chemicals, such as oleylamine, steps for formation of lanthanide trifluoroacetates and the need to filter the unwanted insoluble materials, which will contaminate the reaction medium. Moreover, the high reaction temperature at 310° C. for synthesis of the core-shell NaGdF₄:Yb/Er@NaGdF₄:Yb/Nd is undesirable.

The core multishell structured nanoparticles of NaGdF₄:Yb,Er@NaYF₄:Yb@NaGdF₄:Yb,Nd and NaGdF₄:Yb,Er@NaYF₄:Yb@NaGdF₄:Yb,Nd @NaYF₄@-NaGdF₄:Yb,Tm@NaYF₄ were prepared by co-precipitation method for in vivo imaging (Angew. Chem. Int. Ed. 2016, 128, 2510-2515). The oleate core-UCNPs was first prepared and then purified to grow the multishell UCNPs. The resultant multiple shell UCNPs involved NaGdF₄:Yb/Er as core and NaGdF₄:Yb/Nd as intermediate shell. However, the size of these UCNPs is about 45-85 nm which is too large for fabrication of microarray.

There are a lot of viruses that infect different human organs and cause diseases. Some fatal viral infections have become tremendous public health issues worldwide. Early diagnosis for adequate treatment is therefore essential for fighting viral infections. In view of the above short comings of the existing art and this need for early diagnosis, this invention provides a nanoprobe sandwich assay for nucleotide sequence detection for rapid, accurate and low-cost screening.

### SUMMARY OF THE INVENTION

The present invention relates to nanoprobes and their uses for nucleotide sequence detection.

In one embodiment, this invention provides a kit for detecting oligos having a target nucleic acid sequence, comprising: an upconversion nanoprobe having one or more first oligo probes, each complementary to a first segment of said target nucleic acid sequence, wherein said upconversion nanoprobe can be excited by a near infrared wavelength to emit luminescence at an emission wavelength; and a quenching nanoprobe having one or more second oligo probes, each complementary to a second segment of said target nucleic acid sequence, wherein said quenching nanoprobe can absorb said emission wavelength; wherein when said upconversion nanoprobe and quenching nanoprobe are both bound to the same oligo having said target nucleic acid sequence, said quenching nanoprobe quenches said luminescence from said upconversion nanoprobe.

In another embodiment, this invention provides a method for detecting a target nucleic acid sequence in a sample, comprising the steps of: Preparing a hybridization buffer containing said sample; Adding an upconversion nanoprobe and a quenching nanoprobe to said hybridization buffer to form a mixture; wherein said upconversion nanoprobe has one or more first oligo probes, each complementary to a first segment of said target nucleic acid sequence, wherein said upconversion nanoprobe can be excited by a near infrared wavelength to emit luminescence at an emission wavelength; wherein said quenching nanoprobe has one or more second oligo probes, each complementary to a second segment of said target nucleic acid sequence, wherein said quenching nanoprobe can absorb said emission wavelength; Incubating said mixture for a period of time; and Exposing said mixture to a near infrared wavelength and measuring intensity of said luminescence; wherein when said intensity is lower than intensity of luminescence from a control without said target nucleic acid sequence, said sample is shown to contain said nucleic acid sequence.

In a further embodiment, this invention provides a method for preparing an upconversion nanoprobe, comprising the steps of: Heating a mixture of lanthanide acetates, oleic acid and 1-octadecene; Heating the mixture after the addition of sodium hydroxide and ammonium fluoride; Purifying and precipitating the resulting oleated upconversion nanoparticle by adding an organic solvent; Removing the oleate groups from the oleated upconversion nanoparticle by acid treatment to give ligand-free upconversion nanoparticle (UCNP); Conjugating said UCNP with polyacrylic acid followed by conjugation with an oligo sequence to obtain the upconversion nanoprobe.

### BRIEF DESCRIPTION OF THE DRAWINGS

FIG. 1 illustrates the coprecipitation synthesis of UCNPs in OA and 1-ODE.

FIG. 2 illustrates the instantaneous gold-thiol conjugation via acid-assisted process.

FIG. 3 illustrates the DNA oligo hybridization of the nanoprobes and influenza subtype target.

FIG. 4 shows absorption spectra of AuNPs before and after oligo modification in water.

FIG. 5A shows PCR amplification results of the HA genes.

FIG. 5B shows TEM image of the hybridized sample at 50 pM.

FIG. 5C shows upconversion luminescence spectra at various concentrations at pM range.

FIG. 5D shows quenching efficiency at different concentrations.

### DETAILED DESCRIPTION OF THE INVENTION

The present invention describes a rapid and sensitive detection method that can be easily applied to routine diagnosis. This method simultaneously detects multi specimens.

In one embodiment, this invention provides a kit for detecting oligos having a target nucleic acid sequence, comprising: an upconversion nanoprobe having one or more first oligo probes, each complementary to a first segment of said target nucleic acid sequence, wherein said upconversion nanoprobe can be excited by a near infrared wavelength to emit luminescence at an emission wavelength; and a quenching nanoprobe having one or more second oligo probes, each complementary to a second segment of said target nucleic acid sequence, wherein said quenching nanoprobe can absorb said emission wavelength; wherein when said upconversion nanoprobe and quenching nanoprobe are both bound to the same oligo having said target nucleic acid sequence, said quenching nanoprobe quenches said luminescence from said upconversion nanoprobe.

In one embodiment, said upconversion nanoprobe is selected from a group consisting of NaGdF4:Yb/Er@NaGdF4:Yb/Nd, NaGdF4:Yb/Tm@NaGdF4:Eu, NaGdF4:Yb/Tm@NaGdF4:Tb and NaYF4:Yb/Er@NaYF4:Yb/Nd.

In one embodiment, said quenching nanoprobe uses one of the following as a quencher: gold, Black hole quencher dye, graphene quantum dot or graphene oxide nanosheets.

In one embodiment, said emission wavelength is 520-550 nm.

In one embodiment, said target nucleic acid sequence is any one of SEQ ID. No. 1-8 or part thereof.

In one embodiment, said first oligo probe or second oligo probe is any one of SEQ ID. 9-24 or part thereof.

In one embodiment, said nuclei acid sequence is derived from a group consisting of viruses, viral extracts, bacteria, yeast, fungi, parasites, allergens, cells and cell extracts.

In one embodiment, said viruses are selected from a group consisting of influenza viruses, human immunodeficiency virus/AIDS (HIV/AIDS), hepatitis A virus, hepatitis B virus, hepatitis C virus, hepatitis D virus, hepatitis E virus, Ebola virus, West Nile virus and Zika Virus.

In one embodiment, this invention provides a method for detecting a target nucleic acid sequence in a sample, comprising the steps of: a. Preparing a hybridization buffer containing said sample; b. Adding an upconversion nanoprobe and a quenching nanoprobe to said hybridization buffer to form a mixture; wherein said upconversion nanoprobe has one or more first oligo probes, each complementary to a first segment of said target nucleic acid sequence, wherein said upconversion nanoprobe can be excited by a near infrared wavelength to emit luminescence at an emission wavelength; wherein said quenching nanoprobe has one or more second oligo probes, each complementary to a second segment of said target nucleic acid sequence, wherein said quenching nanoprobe can absorb said emission wavelength; c. Incubating said mixture for a period of time; and d. Exposing said mixture to a near infrared wavelength and measuring the intensity of said luminescence; wherein when said intensity is greater lower than the intensity of luminescence from a control without said target nucleic acid sequence, said sample is shown to contain said nucleic acid sequence.

In one embodiment, said upconversion nanoprobe is selected from a group consisting of NaGdF4:Yb/Er@NaGdF4:Yb/Nd, NaGdF4:Yb/Tm@NaGdF4:Eu, NaGdF4:Yb/Tm@NaGdF4:Tb and NaYF4:Yb/Er@NaYF4:Yb/Nd.

In one embodiment, said quenching nanoprobe uses one of the following as a quencher: gold, Black hole quencher dye, graphene quantum dot or graphene oxide nanosheets.

In one embodiment, said emission wavelength is 520-550 nm.

In one embodiment, said target nucleic acid sequence is any one of SEQ ID. No. 1-8 or part thereof.

In one embodiment, said first oligo probe or second oligo probe is any one of SEQ ID. No. 9-24 or part thereof.

In one embodiment, said nuclei acid sequence is derived from a group consisting of viruses, viral extracts, bacteria, yeast, fungi, parasites, allergens, cells and cell extracts. In another embodiment, said viruses are selected from a group consisting of influenza viruses, human immunodeficiency virus/AIDS (HIV/AIDS), hepatitis A virus, hepatitis B virus, hepatitis C virus, hepatitis D virus, hepatitis E virus, Ebola virus, West Nile virus and Zika Virus.

In one embodiment, this invention provides a method for preparing an upconversion nanoprobe, comprising the steps of: a. Heating a mixture of lanthanide acetates, oleic acid and 1-octadecene; b. Heating the mixture after the addition of sodium hydroxide and ammonium fluoride; c. Purifying and precipitating the resulting oleated upconversion nanoparticle by adding an organic solvent; d. Removing the oleate groups from the oleated upconversion nanoparticle by acid treatment to give ligand-free upconversion nanoparticle (UCNP); and e. Conjugating said UCNP with polyacrylic acid followed by conjugation with an oligo sequence to obtain the upconversion nanoprobe.

In one embodiment, said lanthanide is selected from a group consisting of gadolinium, ytterbium, erbium and neodymium.

In one embodiment, said organic solvent is selected from a group consisting of cyclohexane, methanol, ethanol and any mixture thereof.

In one embodiment, said acid is selected from a group consisting of hydrochloric acid, hydrobromic acid and any mixture thereof.

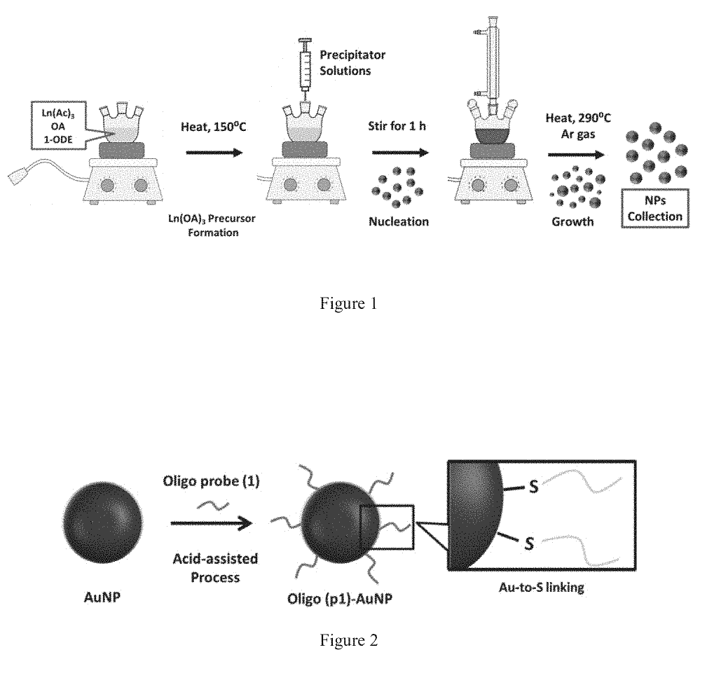

In one embodiment, the invention provides a sandwich assay consisting of an upconversion (UC) and a gold (Au) nanoprobe, in which the target recognition is achieved by two segments of DNA oligonucleotide (oligo) hybridization. The assay increases the specificity towards influenza subtypes because of the dual recognition process. In addition to specificity, UC nanoprobes are used as the luminescent probe because of their near-infrared (NIR) excited nature. This prevents the cross-talking problem because of the large anti-Stokes shift from NIR to visible regime. Moreover, NIR excitation pose minimal damage to biological species compared to high energy ultraviolet light. In addition, Au nanoprobes are selected as the quencher because of its high quenching efficiency. Therefore, the optical signal change can be easily observed by optical detectors. As a result, the close proximity of UC and Au nanoprobe due to oligo hybridization cause the decrease in UC emission intensities via luminescence resonance energy transfer (LRET). The decrement is used to quantify the concentration of influenza subtypes in the sample. The core-shell upconversion nanoparticles (csUCNPs) are chosen because of the flexibility to choose 980 or 808 nm laser excitation source. The NaGdF₄:Yb/Er@NaGdF₄:Yb/Nd csUCNPs are synthesized by the coprecipitation synthesis with oleic acid (OA) and 1-octadecence (1-ODE) as shown FIG. 1. The procedure for the synthesis of core UCNPs started with heating of lanthanide acetates (Ln(AC)₃) with OA and 1-ODE, followed by nucleation and growth under argon (Ar) gas protection. After purification, the same process is repeated for the shell growth by the injection of core UCNPs. The as-synthesized oleate-capped csUCNPs are solubilized by the acid treatment, the hydrochloric acid removes the oleate layer and the ligand-free UCNPs can disperse in water. After that, polyacrylic acid (PAA) is conjugated onto the surface of ligand-free UCNPs via coordination attraction. Then, the PAA-csUCNPs are dispersed in the 2-(N-morpholino)ethanesulfonic acid (MES) buffer with N-Ethyl-N′-(3-dimethylaminopropyl)carbodiimide hydrochloride (EDC.HCl) and N-Hydroxysuccinimide (NHS) for the probe oligo sequence conjugation. The UC nanoprobes can be conjugated with different oligo sequences for specific hybridization with influenza subtypes. The second segment of oligo probe is immobilized onto the surface of citrate-stabilized gold nanoparticles (AuNPs) via an instantaneous conjugation method (FIG. 2). The conventional salting method requires at least a few hours to conjugation DNA oligo to AuNPs via gold-thiol chemisorption while the instantaneous conjugation method simply requires five minute for the conjugation. The conjugation is achieved in the presence of acidic citrate buffer solution. Again, the oligo probe can be modified to suit different target influenza subtypes. After the preparation of the two types of nanoprobes, they are mixed in the hybridization buffer solution (phosphate buffer saline) for two hours for oligo hybridization (FIG. 3). The light intensities of the concentration dependent samples are compared with the control sample for quantification.

The invention will be better understood by reference to the Experimental Details which follow, but those skilled in the art will readily appreciate that the specific experiments are provided only for illustrative purpose, and are not meant to limit the invention scope as described herein, which is defined by the claims following thereafter.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended, and does not exclude additional, un-recited elements or method steps.

### Example 1

### Synthesis of NaGdF4:Yb/Er Core Upconversion Nanoparticles (UCNPs)

0.4 mmol of lanthanide acetates (LnAC3) of gadolinium (Gd3+), ytterbium (Yb3+) and erbium (Er3+) were added to a two-necked flask followed by 4 ml of oleic acid (OA) and 6 ml of 1-octadecene (1-ODE). The mixture was heated to 100° C. for 30 min to remove water. Then, the temperature was maintained at 150° C. for 1 h and cooled to room temperature by removing the heating mantle. 1 mmol of sodium hydroxide (NaOH) and 1.32 mmol of ammonium fluoride (NH4F) in methanol were added to the mixture under stirring. The temperature was maintained at 50° C. for 1 h in the nucleation process. After degassing the mixture for 10 min at 100° C., the mixture was heated at 280° C. for 1.5 h under argon protection. After cooling to room temperature, cyclohexane and ethanol were used to purify and precipitate the oleate UCNPs. Finally, the UCNPs were dispersed in 4 mL cyclohexane for further use.

### Example 2

### Synthesis of NaGdF4:Yb/Er@NaGdF4:Yb/Nd Core-Shell Upconversion Nanoparticles (csUCNPs)

0.4 mmol of lanthanide acetates (LnAC3) of gadolinium (Gd3+), ytterbium (Yb3+) and neodymium (Nd3+) were added to a two-necked flask followed by 4 ml of oleic acid (OA) and 6 ml of 1-octadecene (1-ODE). The mixture was heated to 100° C. for 30 min to remove water. Then, the temperature was maintained at 150° C. for 1 h and cooled to room temperature by removing the heating mantle. The as-dispersed core UCNPs in cyclohexane were injected to the mixture followed by addition of 1 mmol of NaOH and 1.32 mmol of NH4F in methanol was added to the mixture under stirring. The temperature was maintained at 50° C. for 1 h in the nucleation process. After degassing the mixture for 10 min at 100° C., the mixture was heated at 280° C. for 1.5 h under argon protection. After cooling to room temperature, cyclohexane and ethanol were used to purify and precipitate the oleate csUCNPs. Finally, the csUCNPs were dispersed in 4 mL cyclohexane for further use.

### Example 3

### Synthesis of Upconversion Nanoprobe and Gold Nanoprobe

As the coprecipitation synthesis of oleate csUCNPs is given above, this section will focus on the solubilization of the csUCNPs and the instantaneous oligo functionalization of AuNPs.

Polyacrylic Acid (PAA)-Modification of csUCNPs

The oleate groups on the surface of csUCNPs are removed by using hydrochloric acid (HCl) treatment. Briefly, 1 mL of oleate csUCNPs is added to 1 mL 2 M HCl and sonicated for 5 min. Then, high speed centrifugation is applied to extract the ligand-free csUCNPs. The process is repeated for three times until all the brownish color is vanished from the csUCNPs. After that, the ligand-free csUCNPs are dispersed in 3 mL of de-ionized (DI) water with stirring. 1 mL 1 M NaOH is subsequently injected into the mixture and stirred overnight for PAA conjugation. Again, high speed centrifugation in water is performed for three times to purify the PAA-csUCNPs.

### Fabrication of Upconversion Nanoprobe

0.5 mg of PAA-csUCNPs is dispersed in 2-(N-morpholino)ethanesulfonic acid (MES) buffer with 0.5 mg of (3-Dimethylaminopropyl)-N′-ethylcarbodiimide hydrochloride (EDC.HCl) and 1.5 mg of N-Hydroxysuccinimide (NHS). The mixture is shaken for 20 mM for activation of surface. Then, 0.5 nmol oligo probe is injected into the mixture and conjugated for 2 h. Then, the upconversion nanoprobe is purified by centrifugation in water.

### Fabrication of Gold Nanoprobe

The citrate-stabilized AuNPs are obtained from Sigma-Aldrich. 10 μL thiolated oligo probe is added to 200 μL AuNPs. Then, 2 μL of 50 mM phosphate buffer and 10 μL of citrate buffer are added to the tube of mixture. After vortexing the mixture, the gold-thiol conjugation is allowed to react for 8 min. Finally, the gold nanoprobe is collected by high speed centrifugation for 10 mM.

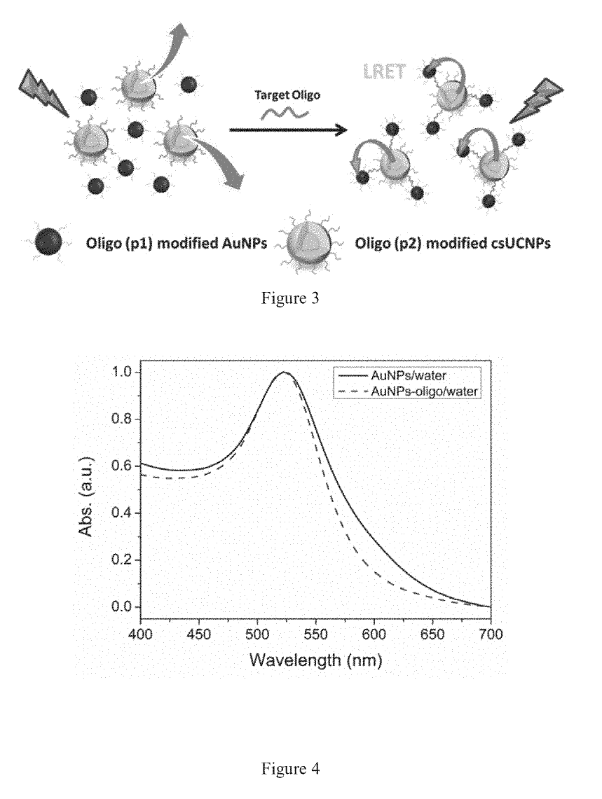

In the present approach, the AuNPs are modified by using a thiolated oligo sequence via an acid-assisted approach.^{[1]} The modification is completed in 10-15 min and it is referred as an instantaneous modification. The process also started with the citrate-stabilized gold nanoparticles and thiolated oligos; however the addition of citrate buffer speeds up the gold-thiol reaction (FIG. 2). Moreover, the oligo sequences are specific to the sequences provided in the World Health Organization standard protocol for detection of influenza virus subtypes. Therefore, this justified the enhanced specificity. The quality of the modification can be reflected in the UV-vis spectroscopy. FIG. 4 shows the absorption spectra of the AuNPs before and after the oligo modification. The absorption maxima indicated no shift in wavelength; this suggests the AuNPs-oligo had no aggregation with high stability in water.

### Example 4

### Detection of Nucleotide Sequences

To improve the specificity and efficiency of detection, the upconversion sandwich assay was chosen (FIG. 3) to increase the specificity and efficiency of detection. In the present assay, the csUCNPs and oligo modifications are used to form the first probe (P2) but the acid-assisted approached is used to conjugate another segment of oligo probe (P1) on AuNPs. Therefore, two types of nanoprobes are prepared for detection: csUCNPs-probe and AuNPs-probe. The influenza subtype virus oligos are added to the hybridization medium with the probes for 0.5-2 h followed by the readout process. The sequences are obtained from the standard protocol for influenza detection published by the World Health Organization.

### Example 5

### Detection of Multiple Targets

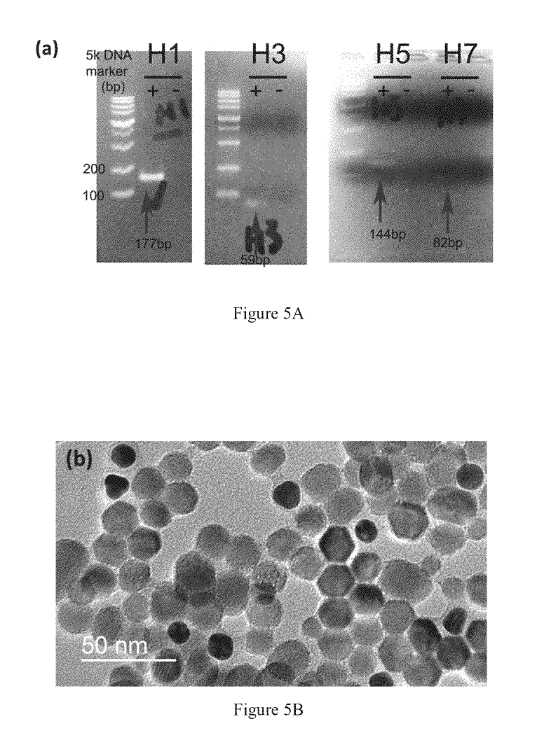

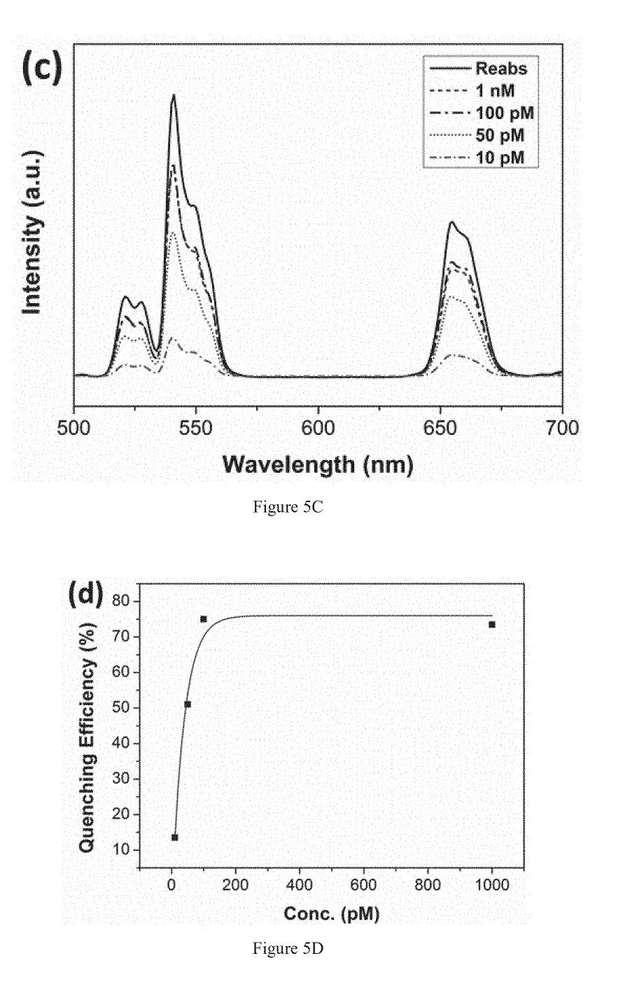

Clinical samples of HA genes were obtained from a hospital and validated by RT-PCR. The validation had been achieved by gel electrophoresis as shown in FIG. 5A. The results indicated the positive readout of subtypes H1, H3, H5 and H7 with different base-pairs (bp). In addition, the spatial distribution of UCNPs-probe and AuNPs-probes after hybridize with the clinical samples is shown in FIG. 5B. The electron micrograph shows the spherical and hexagonal-shaped csUCNPs-probes and the dark AuNPs-probes. The AuNPs-probes are bounded to the csUCNPs-probe because of oligo hybridization. Moreover, thin layer of shell can be observed on the surface of the csUCNPs-probe and AuNPs-probe, which corresponds to the layer of conjugated oligo probe. FIGS. 5C and 5D presents the H1 gene detection using the upconversion sandwich assay at various concentrations. The quenching efficiency can be attained as high as 75% and the lowest detectable concentration is around 10 picomolar (pM). Importantly, the detection scheme exhibit rapid and highly specific detection.

### Example 6

### Target Sequences and Probes

In this invention, the detection of viral subtype genes is achieved by using the upconversion nanoprobe sandwich assay. The following target influenza virus sequences are given by the World Health Organization in the article titled “WHO information for molecular diagnosis of influenza virus—update”:

H1:
(SEQ ID NO: 1)
5′-GGGGTAGCCCCATTGCATTTGGGTAA-3′ (26 bases)
H3:
(SEQ ID NO: 2)
5′-AACAGTTGCTGTAGGCTTTGCTGCGT-3′ (26 bases)
H5:
(SEQ ID NO: 3)
5′-TGGATTCTTTGTCTGCAGCGTACCCA-3′ (26 bases)
H7:
(SEQ ID NO: 4)
5′-CCGCTGCTTAGTTTGACTGGGTCA-3′ (24 bases)
N1:
(SEQ ID NO: 5)
5′-ATGTTGAACGAAACTTCCGCTG-3′ (22 bases)
N2:
(SEQ ID NO: 6)
5′-TGTGGAGTTGATAAGGGGAAG-3′ (21 bases)
N8:
(SEQ ID NO: 7)
5′-TGACCAGTCGGCAATCTCATAGT-3′ (23 bases)
N9:
(SEQ ID NO: 8)
5′-GGGTCATTCGGTCGGGGATTGTCT-3′ (24 bases)

In the upconversion nanoprobe sandwich assay, the target is captured by the strands of two oligo probe sequences. The first oligo probe sequence is conjugated onto PAA-csUCNPs while the second is conjugated onto AuNPs.

Some examples of the probe sequences on the PAA-csUCNPs are listed below:

H1:
(SEQ ID NO: 9)
5′-TTACCCAAATGCA-3′ (13 bases)
H3:
(SEQ ID NO: 10)
5′-ACGCAGCAAAGCC-3′ (13 bases)
H5:
(SEQ ID NO: 11)
5′-TGGGTACGCTGCA-3′ (13 bases)
H7:
(SEQ ID NO: 12)
5′-TGACCCAGTCAA-3′ (12 bases)
N1:
(SEQ ID NO: 13)
5′-CAGCGGAAGTT-3′ (11 bases)
N2:
(SEQ ID NO: 14)
5′-CTTCCCCTTAT-3′ (11 bases)
N8:
(SEQ ID NO: 15)
5′-ACTATGAGATTG-3′ (12 bases)
N9:
(SEQ ID NO: 16)
5′-AGACAATCCCCG-3′ (12 bases)

Some examples of the probe sequences on the AuNPs are listed below:

H1:
(SEQ ID NO: 17)
5′-ATGGGGCTACCCC-3′ (13 bases)
H3:
(SEQ ID NO: 18)
5′-TACAGCAACTGTT-3′ (13 bases)
H5:
(SEQ ID NO: 19)
5′-GACAAAGAATCCA-3′ (13 bases)
H7:
(SEQ ID NO: 20)
5′-ACTAAGCAGCGG-3′ (12 bases)
N1:
(SEQ ID NO: 21)
5′-TCGTTCAACAT-3′ (11 bases)
N2:
(SEQ ID NO: 22)
5′-CAACTCCACA-3′ (10 bases)
N8:
(SEQ ID NO: 23)
5′-CCGACTGGTCA-3′ (11 bases)
N9:
(SEQ ID NO: 24)
5′-ACCGAATGACCC-3′ (12 bases)

### REFERENCE

1. X. Zhang, M. R. Servos, J. Liu, J. Am. Chem. Soc. 2012, 134, 7266.

What is claimed is:

1. A kit for detecting oligos having a target nucleic acid sequence, comprising:
a. An upconversion nanoprobe having one or more first oligo probes, each complementary to a first segment of said target nucleic acid sequence, wherein said upconversion nanoprobe can be excited by a near infrared wavelength to emit luminescence at an emission wavelength; and
b. A quenching nanoprobe having one or more second oligo probes, each complementary to a second segment of said target nucleic acid sequence, wherein said quenching nanoprobe can absorb said emission wavelength;
wherein when said upconversion nanoprobe and quenching nanoprobe are both bound to the same oligo having said target nucleic acid sequence, said quenching nanoprobe quenches said luminescence from said upconversion nanoprobe.

2. The kit of claim 1, wherein said upconversion nanoprobe is selected from a group consisting of NaGdF₄:Yb/Er@NaGdF₄:Yb/Nd, NaGdF4:Yb/Tm@NaGdF4:Eu, NaGdF4:Yb/Tm@NaGdF4:Tb and NaYF4:Yb/Er@NaYF4:Yb/Nd.

3. The kit of claim 1, wherein said quenching nanoprobe uses one of the following as a quencher: gold, Black hole quencher dye, graphene quantum dot or graphene oxide nanosheets.

4. The kit of claim 1, wherein said emission wavelength is 520-550 nm.

5. The kit of claim 1, wherein said target nucleic acid sequence is any one of SEQ ID. No. 1-8 or part thereof.

6. The kit of claim 1, wherein said first oligo probe or second oligo probe is any one of SEQ ID. No. 9-24 of part thereof.

7. The kit of claim 1, wherein said nuclei acid sequence is derived from a group consisting of viruses, viral extracts, bacteria, yeast, fungi, parasites, allergens, cells and cell extracts.

8. The kit of claim 7, wherein said viruses are selected from a group consisting of influenza viruses, human immunodeficiency virus/AIDS (HIV/AIDS), hepatitis A virus, hepatitis B virus, hepatitis C virus, hepatitis D virus, hepatitis E virus, Ebola virus, West Nile virus and Zika Virus.

9. A method for detecting a target nucleic acid sequence in a sample, comprising the steps of:
a. Preparing a hybridization buffer containing said sample;
b. Adding an upconversion nanoprobe and a quenching nanoprobe to said hybridization buffer to form a mixture; wherein said upconversion nanoprobe has one or more first oligo probes, each complementary to a first segment of said target nucleic acid sequence, wherein said upconversion nanoprobe can be excited by a near infrared wavelength to emit luminescence at an emission wavelength; wherein said quenching nanoprobe has one or more second oligo probes, each complementary to a second segment of said target nucleic acid sequence, wherein said quenching nanoprobe can absorb said emission wavelength;
c. Incubating said mixture for a period of time; and
d. Exposing said mixture to a near infrared wavelength and measuring intensity of said luminescence;
wherein when said intensity is lower than the intensity of luminescence from a control without said target nucleic acid sequence, said sample is shown to contain said nucleic acid sequence.

10. The method of claim 9, wherein said upconversion nanoprobe is selected from a group consisting of NaGdF₄:Yb/Er@NaGdF₄:Yb/Nd, NaGdF4:Yb/Tm@NaGdF4:Eu, NaGdF4:Yb/Tm@NaGdF4:Tb and NaYF4:Yb/Er@NaYF4:Yb/Nd.

11. The method of claim 9, wherein said quenching nanoprobe uses one of the following as a quencher: gold, Black hole quencher dye, graphene quantum dot or graphene oxide nanosheets.

12. The method of claim 9, wherein said emission wavelength is 520-550 nm.

13. The method of claim 9, wherein said target nucleic acid sequence is any one of SEQ ID. No. 1-8 or part thereof.

14. The method of claim 9, wherein said first oligo probe or second oligo probe is any one of SEQ ID. No. 9-24 or part thereof.

15. The method of claim 9, wherein said nuclei acid sequence is derived from a group consisting of viruses, viral extracts, bacteria, yeast, fungi, parasites, allergens, cells and cell extracts.

16. The method of claim 15, wherein said viruses are selected from a group consisting of influenza viruses, human immunodeficiency virus/AIDS (HIV/AIDS), hepatitis A virus, hepatitis B virus, hepatitis C virus, hepatitis D virus, hepatitis E virus, Ebola virus, West Nile virus and Zika Virus.

17. A method for preparing an upconversion nanoprobe, comprising the steps of:
a. Heating a mixture of lanthanide acetates, oleic acid and 1-octadecene;
b. Heating the mixture after the addition of sodium hydroxide and ammonium fluoride;
c. Purifying and precipitating the resulting oleated upconversion nanoparticle by adding an organic solvent;
d. Removing the oleate groups from the oleated upconversion nanoparticle by acid treatment to give ligand-free upconversion nanoparticle (UCNP); and
e. Conjugating said UCNP with polyacrylic acid followed by conjugation with an oligo sequence to obtain said upconversion nanoprobe.

18. The method of claim 17, wherein said lanthanide is selected from a group consisting of gadolinium, ytterbium, erbium, europium, thulium and neodymium.

19. The method of claim 17, wherein said organic solvent is selected from a group consisting of cyclohexane, toluene and any mixture thereof.

20. The method of claim 17, wherein said acid is selected from a group consisting of hydrochloric acid, hydrobromic acid and any mixture thereof.
